(19) United States
(12) Patent Application Publication
(10) Pub. No.: US 2016/0128597 A1
(43) Pub. Date: May 12, 2016

(54) THIN PLANAR BIOLOGICAL SENSOR

(71) Applicant: NATIONAL CHIAO TUNG UNIVERSITY, HSINCHU CITY (TW)

(72) Inventors: CHIN-TENG LIN, HSINCHU COUNTY (TW); YI-HSIN YU, HSINCHU CITY (TW); SHAO-WEI LU, HSINCHU CITY (TW); LUN-DE LIAO, TAICHUNG CITY (TW)

(21) Appl. No.: 14/845,720

(22) Filed: Sep. 4, 2015

(30) Foreign Application Priority Data
Nov. 11, 2014 (TW) 103139019

(51) Int. Cl. A61B 5/0478; A61B 5/0496; A61B 5/0488; A61B 5/00; A61B 5/0402

(52) U.S. Cl. CPC A61B 5/0478; A61B 5/6814; A61B 5/0402; A61B 5/0488; A61B 5/0496; A61B 5/0006

(57) ABSTRACT

A thin planar biological sensor is provided, comprising at least one flexible electrode used for contacting a subject's skin and measuring biological signal. The flexible electrode is made of silicon-silver-based material, in which a ratio of silicon to silver-compound is about 7:3. A conductive plate is coupled to the flexible electrode. A buffer layer covers the conductive plate and the flexible electrode. A printed circuit board is used to receive the measured biological signal. By employing the novel proposed biological sensor, it is advantageous of measuring biological signals not only EEG signals but also ECG and EMG signals. Besides being thin, planar, flexible and non-allergy attributing a low cost to the proper fabrications makes the proposed sensor characterized with a potential for becoming an important tool of medical measurement.

This application claims priority for Taiwan patent application no. 103139019 filed on Nov. 11, 2014, the content of which is incorporated by reference in its entirely.

### BACKGROUND OF THE INVENTION

1. Field of the Invention

The present invention relates to a biological sensor, and more particularly to a thin planar biological sensor which adopts silicon-silver-based conductive material that is both flexible and biologically-compatible as measuring electrodes.

2. Description of the Related Art

Biomedical measurement systems are popular medical apparatuses. Many research papers have been proposed to improve the inconveniences and drawbacks of biomedical measurement systems nowadays. Traditional EEG (electroencephalography) systems normally intend to adopt wet electrodes. However, it is well known that wet electrodes have to cooperate with conductive glue, and the conductive glue may cause discomfort or allergy to patients. Besides, conductivity of the conductive glue decays with time. Similar to the electrodes of EEG systems, the electrodes of biomedical measurement systems also need conductive glue and have the same drawbacks of wet electrodes.

Dry electrodes offer another choice to be adopted since they are more convenient than wet electrodes to use. However, at present, dry electrodes are almost fabricated in microstructure processes, such as the MEMS (micro-electromechanical system) process and the carbon nano-tube process. It is believed that these microstructure-based dry electrodes are kind of invasive measurements, and are likely to fracture and hard to apply to hairy regions of a subject. Therefore is impaired popularization of dry electrodes.

Moreover, since biological measurements and biomedical science have attracted more and more attention in recent years, thus improvements and applications of biomedical measurement apparatuses have gradually become an important subject. The current tendency is to miniaturize apparatuses and realize instant and long-term biomedical measurement. The conventional bulky and complicated devices should have been out of date. However, many researches are still impeded by shortage of efficacious and cost-efficient technologies, thereby limiting more and more developments still until now.

As a result, on account of all, it should be obvious that there is indeed an urgent need for the professionals in the field for a new biological sensor to be developed that is totally different from the previous wet electrodes and dry electrodes used before so as to solve the above-mentioned problems occurring in the prior art.

### SUMMARY OF THE INVENTION

In order to overcome the above-mentioned disadvantages, one primary objective of the present invention is to provide a thin planar biological sensor, which not only successfully solves the conventional issues of the prior measuring electrodes, but also makes it possible to perform instantaneous and extended biological measurements. By employing the proposed thin planar biological sensor, the measured biological signals can also be more stable and will not decay from time to time.

Another objective of the present invention is to provide a thin planar biological sensor, which adopts silicon-silver-based conductive material that is both flexible and biologically-compatible as measuring electrodes. Together with an injection molding process being used to fabricate the measuring electrodes, the measuring electrodes are quite flexible, and therefore provide better adhesion and still excellent measuring properties to the subject even when the subject may be in a moving status.

A further objective of the present invention is to provide a thin planar biological sensor, wherein the measuring electrodes of the present invention are exempted from involving with the conductive glue and therefore avoiding the drawbacks of the conventional wet electrodes. Moreover, the proposed thin planar biological sensor is able to be applied to measure not only EEG signals but also other biomedical signals, such as ECG, EMG and EOG signals, thereby functioning as a widely used mainstream instrument in biomedical measurement.

To achieve the abovementioned objectives, the present invention proposes a thin planar biological sensor which comprises at least one flexible electrode, at least one conductive plate, a buffer layer and a printed circuit board. The flexible electrode is used for contacting a subject's skin and measuring biological signals of the subject, wherein the flexible electrode is made of silicon-silver-based material, in which a ratio of silicon to silver-compound is about 7:3. The conductive plate is electrically coupled to the flexible electrode, and disposed below the flexible electrode. The buffer layer is further disposed below the conductive plate such that the conductive plate is disposed between the buffer layer and the flexible electrode. The printed circuit board is further disposed below the buffer layer and electrically coupled to the conductive plate and the flexible electrode for receiving the biological signals of the subject.

According to one embodiment of the present invention, the silver-compound further comprises silver and silicon-dioxide (SiO₂), in which a ratio of silver to silicon-dioxide is 1:9. Furthermore, the printed circuit board of the present invention is selected to be a flexible PCB. As such, since the flexible electrode, the flexible PCB and the buffer layer of the present invention are all easy to perform deformation, it makes the proposed biological sensor full of more flexibility such that the proposed biological sensor is able to show admissible deformation according to different cave or prominence of the subject. Also, it still maintains extraordinary measuring properties even when the subject is in a moving status.

Moreover, the measured biological signals of the present invention can be further transmitted to a Tx/Rx (signal transmitting and receiving element) through at least one wire connected to the printed circuit board. Thus, the Tx/Rx is able to send the biological signals to a remote end for further analyses. As a result, it is apparent that based on the techniques the present invention discloses, the flexible electrode contacts the subject's skin for rapidly obtaining his or her biological signals. Since the whole biological sensor is very flexible, the electrode is much easier to be in good contact with the subject's skin so as to achieve the best measuring efficiency.

In addition, since the present invention dramatically simplifies the conventional sensor structure and fabrication process of the prior art, it is believed to stand as one of the major appliances developed in the future for biomedical measurements. Below, embodiments are described in detail in cooperation with drawings to make easily understood the objectives, technical contents, characteristics and accomplishments of the present invention.

Also, these and other objectives of the present invention will become obvious to those of ordinary skill in the art after reading the following detailed description of preferred embodiments.

It is to be understood that both the foregoing general description and the following detailed description are exemplary, and are intended to provide further explanation of the invention as claimed.

### BRIEF DESCRIPTION OF THE DRAWINGS

The accompanying drawings are included to provide a further understanding of the invention, and are incorporated in and constitute a part of this specification. The drawings illustrate embodiments of the invention and, together with the description, serve to explain the principles of the invention. In the drawings:

FIG. 1 shows a top view of a thin planar biological sensor according to one embodiment of the present invention;

FIG. 2 shows a side view of a thin planar biological sensor according to one embodiment of the present invention;

FIG. 3 shows a front view of a thin planar biological sensor according to one embodiment of the present invention;

FIG. 4 shows a back view of a thin planar biological sensor according to one embodiment of the present invention;

FIG. 5 shows a schematic drawing of a thin planar biological sensor according to one embodiment of the present invention while in use;

FIG. 6 shows a side drawing of a subject wearing the elastic belt according to one embodiment of the present invention as shown in FIG. 5;

FIG. 7 shows test results of the conventional wet electrodes used for measuring a subject's forehead signals;

FIG. 8 shows test results of the proposed invention used for measuring a subject's forehead signals; and

FIG. 9 shows test results of the measured resistance of the thin planar biological sensor of the present invention.

### DETAILED DESCRIPTION OF THE INVENTION

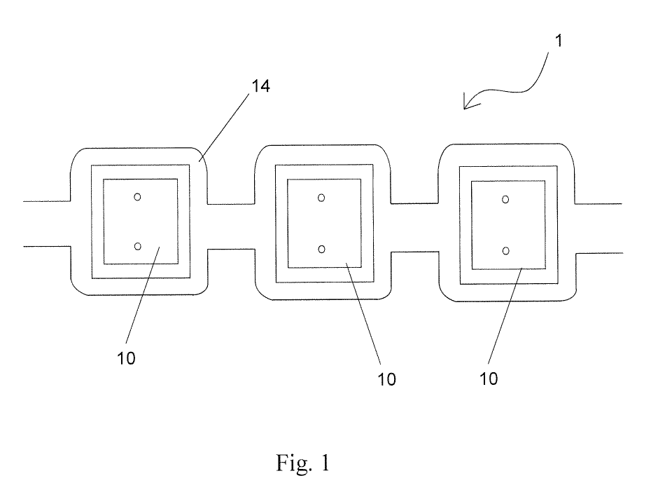

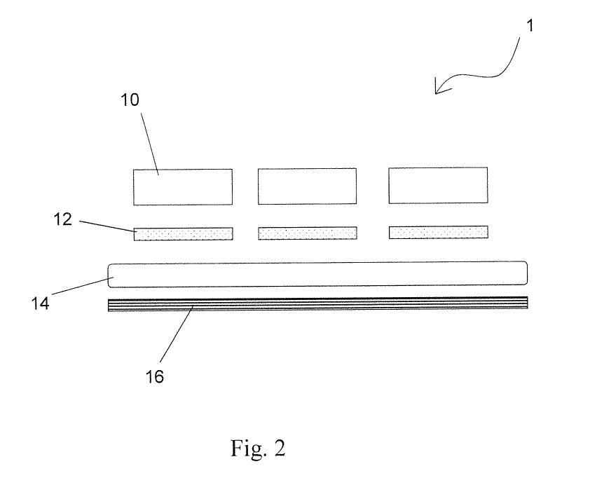

Refer to FIG. 1 and FIG. 2, which respectively shows a top view and a side view of a thin planar biological sensor according to one embodiment of the present invention. As shown, the thin planar biological sensor 1 comprises at least one flexible electrode 10, at least one conductive plate 12, a buffer layer 14 and a printed circuit board 16, wherein the flexible electrode 10 is made of silicon-silver-based material, that is both bendable, conductive, and non-allergy. According to the present invention, a ratio of the silicon to silver-compound is about 7:3. And, the silver-compound further comprises silver and silicon-dioxide (SiO₂), in which a ratio of silver to silicon-dioxide is 1:9. Since the proposed silver-compound has great conductivity, and shows extinct flexibility while combined with SiO₂, it helps to make the proposed biological sensor much more flexible and cozy than ever. Therefore, when the thin planar biological sensor of the present invention is placed on a subject's skin and fixed by external machinery, it can be effectively utilized for measuring biological signals of the subject. Also, owing to the well-conductive material, the silicon-silver-based material the present invention is characterized by, the proposed electrode is able to detect not only EEG (electroencephalography), ECG (electrocardiography), EMG (electromyography) and EOG (electrooculography) signals, but also to detect very weak signals with a very high sensitivity without using conductive glue, whereby the operation is more convenient and efficient.

The conductive plate 12 is electrically coupled to the flexible electrode 10 and disposed below the flexible electrode 10. In order to build the electrical connection, the conductive plate 12 of the present invention is made of metal, for example. For the objectives of performing independent EEG signals analyses and multi-channels measurements, the number of the flexible electrode 10 the present invention uses might be sixteen, thirty-two, or sixty-four. Under such circumstances, the number of the conductive plate 12 can be either one or more than one. In other words, the numbers of the flexible electrode 10 connected to the conductive plate 12 can be designed as one-to-one or multiple-to-one. FIG. 2 merely shows one of the embodiments of the present invention, however, the present invention is not limited thereto.

Moreover, as shown in FIG. 1 and FIG. 2, the buffer layer 14 is disposed below the conductive plate 12 such that the conductive plate 12 is disposed between the flexible electrode 10 and the buffer layer 14. According to the embodiment of the present invention, the buffer layer 14 can be made of soft silicon material so as to cover the flexible electrode 10 and the conductive plate 12. Therefore, based on both the soft silicon material of the buffer layer and the great flexibility of the electrode, it helps to make the whole biological sensor much more flexible, and much more adhesive to the subject's skin while measurements are done more precisely. In addition, the subject may enjoy better comfort and sensation because of the soft silicon he or she is being contact with.

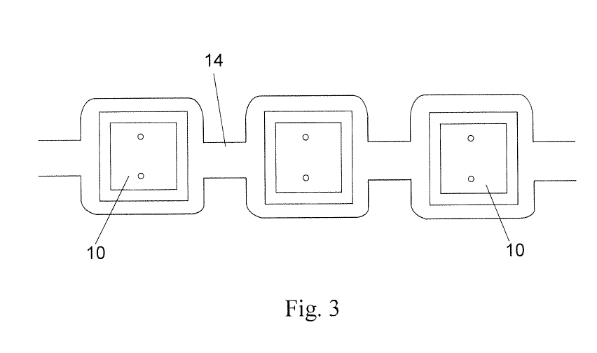

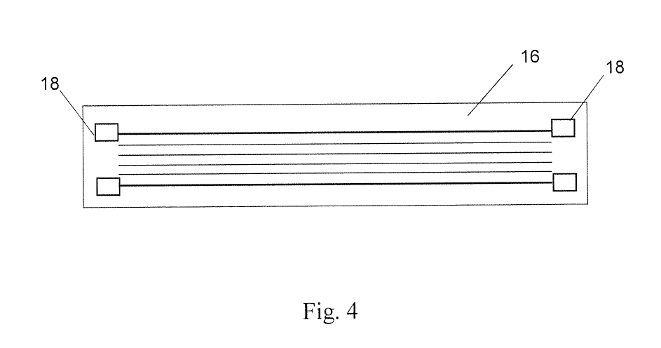

The printed circuit board 16 is disposed below the buffer layer 14 and electrically connected to the conductive plate 12 and the flexible electrode 10. As such, when the flexible electrode 10 is placed upon the subject, for example, the subject's head to measure biological signals (i.e. EEG signals), the measured signals can be transmitted to and received by the printed circuit board 16. Refer to FIG. 3 and FIG. 4, which respectively shows a front view and a back view of a thin planar biological sensor according to one embodiment of the present invention. As shown, there are a plurality of wire disposed upon the printed circuit board 16, which are electrically connected to the silicon-silver-based electrode. An output port 18 is further disposed on another side of the printed circuit board. As a result, by employing these wires or signal transmitting lines connected thereto, the proposed biological sensor is able to successfully measure and transmit the biological signals.

According to one embodiment of the present invention, the printed circuit board 16 for example, can be a flexible printed circuit (FPC) for providing better comfort to the subject. As a result, since the proposed flexible electrode, FBC and silicon buffer payer of the present invention all have great flexibility, it is believed that the biological sensor composed thereof is extraordinary flexible as well. When it is applied to the subject's skin, it can perform excellent deformation based on different cave or protuberance the subject's skin is showing and meanwhile still maintain very good measuring properties.

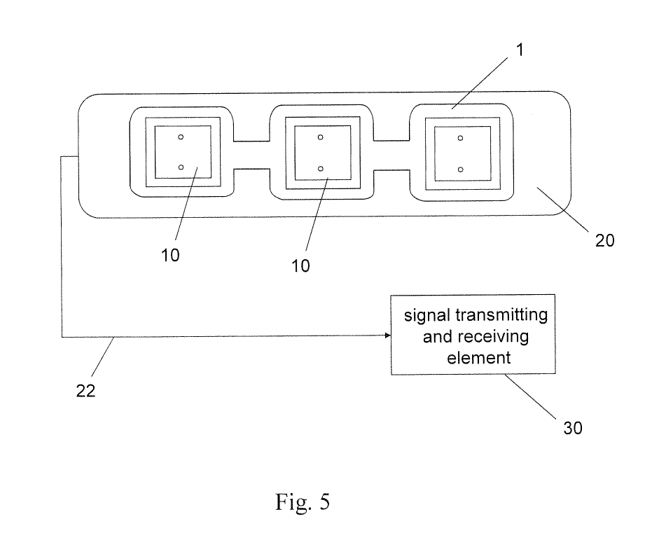

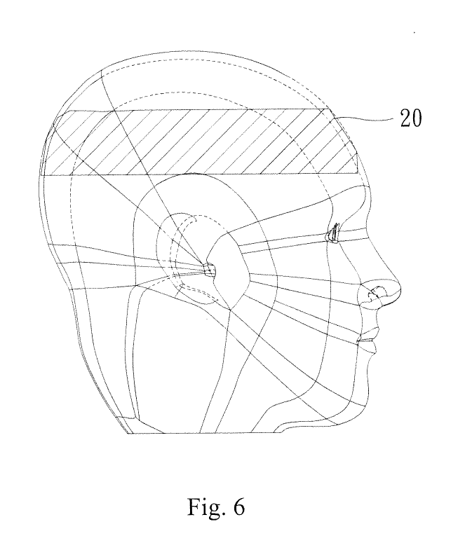

Please refer to FIG. 5, which shows a schematic drawing of a thin planar biological sensor according to one embodiment of the present invention while in use. As shown, the thin planar biological sensor 1 of the present invention can be further disposed on an elastic belt 20. Therefore, while in use as shown in FIG. 6, the subject may directly wear the elastic belt 20 over his or her head, and the elastic belt 20 surrounds the head such that the flexible electrode 10 disposed inside the elastic belt 20 can connect the subject's head much more adhesively, thereby increasing precision of the measurement results. In addition, by employing the belt surrounding the subject's head, the proposed biological sensor is able to maintain good measuring properties even when the subject is in a moving status without causing any measurement errors.

Later, the biological signals measured by the thin planar biological sensor 1 of the present invention can be further transmitted to a signal transmitting and receiving element 30 through at least one wire 22. As such, the signal transmitting and receiving element 30 receives the measured biological signals, and further transmits them again to a remote end. In one embodiment, the signal transmitting and receiving element 30 is connected to the remote end through a wireless connection, for example Wifi, WiMax, or Bluetooth (BT). Furthermore, in order to eliminate electromagnetic interferences (EMI) of the present invention, a housing can be further disposed outside the signal transmitting and receiving element 30 and the housing is made of anti-electrostatic and anti-electromagnetic material. As such, according to the embodiment of the present invention, since the housing is made of anti-electrostatic and anti-electromagnetic material and is made by an injection molding process, the above mentioned electrode and conductive plate can both be directly placed in the mold and be formed at one time. Moreover, for making the whole sensor much more compact and intense, the thin planar biological sensor 1 of the present invention can further comprise conductive paste inside its structure for bonding the flexible electrode 10, the conductive plate 12, the buffer layer 14 and the printed circuit board 16 together and increase strength of its structure. On the other hand, the conductive paste also helps to enhance conductivity of the whole sensor structure for providing better accuracy of measurement results.

As a result, to sum up, since traditional EEG (electroencephalography) systems normally intend to utilize wet electrodes, which have to cooperate with conductive glue, the conductive glue is known to cause discomfort or allergy to patients, and also conductivity of the conductive glue decays with time, nevertheless dry electrodes mostly fabricated in microstructure processes, such as MEMS are invasive measurements, which are likely to fracture and hard to apply to hairy regions, compared to them, the present invention indeed provides a novel biological sensor, which adopts silicon-silver-based conductive material that is not only flexible, biologically-compatible, but also non-allergy and easy to operate, as measuring electrodes, thereby avoiding a great number of drawbacks of the conventional electrodes used in the prior art. As such, it is believed that the present invention apparently possesses as many advantages of operation convenience and measurement precision.

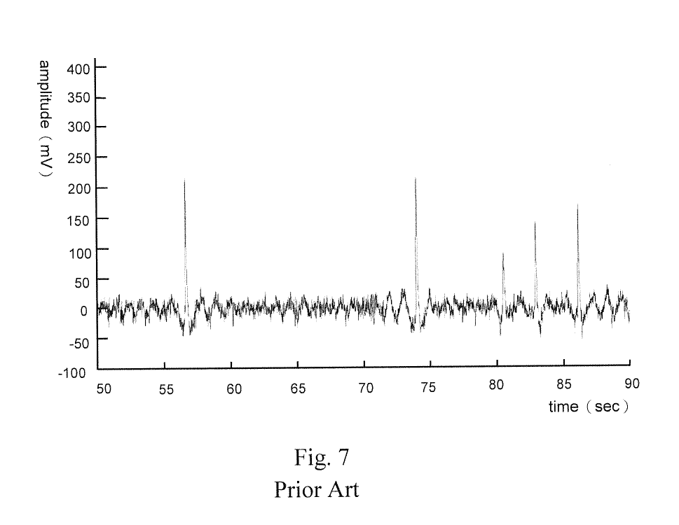

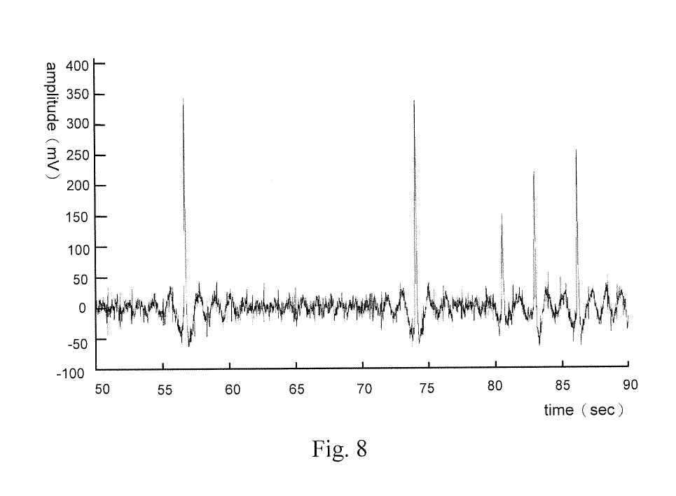

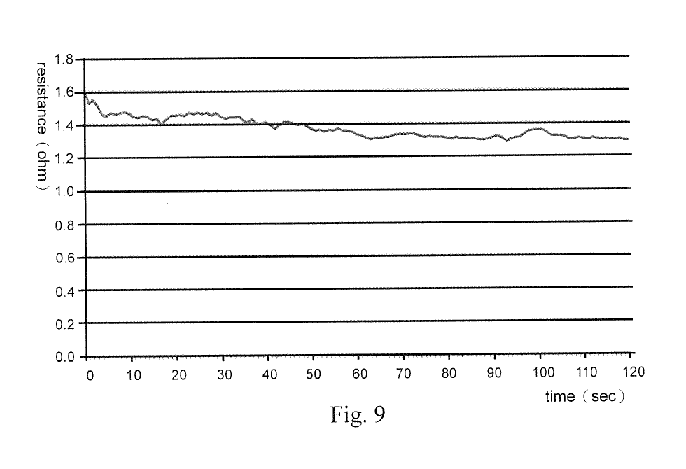

Next, the present invention is verified with implementing test structures to examine the experimental results, which are shown as FIG. 7 and FIG. 8, which respectively shows test results of the conventional wet electrodes and test results of the proposed invention. As the organized results shown in these two figures, it is apparent that the measured biological signals (forehead signals of same subject) are almost the same. Referring to correlation coefficient calculated by MATLAB, the result is about 0.9200, indicating the correlation between these two is quite high. Therefore, it is well proved that the thin planar biological sensor of the present invention is indeed effective and able to offer almost the same measured signals as the conventional wet electrodes did. Furthermore, please refer to FIG. 9, which shows test results of the measured resistance of the thin planar biological sensor of the present invention. As shown, it is obvious that the resistance of the proposed biological sensor regardless of being contact with the subject's skin or being measured single, is quite low (average resistance=1.3 ohm), thereby proving that the proposed sensor is indeed applicable to measure human biological signals.

Therefore, as above mentioned, the present invention discloses a thin planar biological sensor which has never been provided or applied to the like products ever. According to the present invention, no conductive glue is needed, thereby avoiding a great number of prior issues which occurred in the past when conductive glue is a must. Also, the present invention can apply to measure biological signals even when the subject is in a moving status, which solves the prior problems when traditional measurements can also be done during the subject is immobile. Moreover, size of the flexible electrode of the present invention is not limited. In other words, the proposed electrode of the present invention can be adjustable according to different needs, and thus having better flexibility and practical utility.

Moreover, the thin planar biological sensor of the present invention is made disposable, which can be washed out through soapsuds, and sterilized by using hydrogen peroxide (H₂O₂) solution. As such, the price thereof can be dramatically reduced in mass-production, making the proposed biological sensor much more competitive in the market. As a result, it is believed that the present invention truly provides instant and long-term biomedical measurements for subjects and is characterized by convenient operation and real-time supervision. Therefore, all the above mentioned advantages would make the biological sensor of the present invention one of the mainstream instruments developed in future biomedical researches and measurements.

The embodiments described above mentioned are only to exemplify the present invention to enable the persons skilled in the art to understand, make, and use the present invention. However, it is not intended to limit the scope of the present invention. Any equivalent modification or variation according to the spirit of the present invention is to be also included within the scope of the present invention.

Those skilled in the art are able to make various modifications and variations to the present invention without departing from the scope or spirit of the invention. In view of the foregoing, it is intended that the present invention cover modifications and variations of this invention provided they fall within the scope of the invention and its equivalent.

What is claimed is:

1. A thin planar biological sensor, which applies to measuring biological signals of a subject, comprising
at least one flexible electrode used for contacting said subject's skin and measuring biological signals of said subject, wherein said flexible electrode is made of silicon-silver-based material, in which a ratio of silicon to silver-compound is 7:3;
at least one conductive plate, electrically coupled to said flexible electrode and disposed below said flexible electrode;
a buffer layer, disposed below said conductive plate such that said conductive plate is disposed between said buffer layer and said flexible electrode; and
a printed circuit board, disposed below said buffer layer and electrically coupled to said conductive plate and said flexible electrode for receiving biological signals of said subject.

2. The thin planar biological sensor according to claim 1, wherein said silver-compound further comprises silver and silicon-dioxide (SiO₂), in which a ratio of silver to silicon-dioxide is 1:9.

3. The thin planar biological sensor according to claim 1, wherein said conductive plate is made of metal.

4. The thin planar biological sensor according to claim 1, wherein said buffer layer is made of soft silicon so as to cover said flexible electrode and said conductive plate.

5. The thin planar biological sensor according to claim 1, further comprising a signal transmitting and receiving element, which is connected to said printed circuit board through at least one wire so as to receive biological signals of said subject and to transmit said biological signals to a remote end for further analyses.

6. The thin planar biological sensor according to claim 5, wherein said signal transmitting and receiving element is connected to said remote end through a wireless connection.

7. The thin planar biological sensor according to claim 5, further comprising a housing disposed outside said signal transmitting and receiving element, and said housing is made of anti-electrostatic and anti-electromagnetic material.

8. The thin planar biological sensor according to claim 7, wherein said housing is made by an injection molding process.

9. The thin planar biological sensor according to claim 1, wherein said printed circuit board is selected to be a flexible printed circuit (FPC).

10. The thin planar biological sensor according to claim 1, wherein said biological signals of said subject can be selected from a group consisting of EEG, ECG, EMG, and EOG signals.

11. The thin planar biological sensor according to claim 1, further disposed on an elastic belt, in which said elastic belt surrounds said subject's head such that said flexible electrode contacts said subject's head much more adhesively.

12. The thin planar biological sensor according to claim 1, wherein a number of said flexible electrode can be one or more than one, and a number of said conductive plate connected to said flexible electrode can be one or more than one.
